(19) United States
(12) Patent Application Publication
(10) Pub. No.: US 2026/0108433 A1
(43) Pub. Date: Apr. 23, 2026

(54) Plant Glycoside Microcapsule, and Preparation Method and Application

(71) Applicants: Infinitus (China) Company Ltd., Jiangmen (CN); South China University of Technology, Guangzhou (CN)

(72) Inventors: Lei XIAO, Jiangmen (CN); Jinlian HUANG, Jiangmen (CN); Xiaoxi LI, Jiangmen (CN); Tengfei QU, Jiangmen (CN); Lulu DONG, Jiangmen (CN)

(21) Appl. No.: 19/333,193

(22) Filed: Sep. 18, 2025

(30) Foreign Application Priority Data
Oct. 18, 2024 (CN) 202411456569.9

(51) Int. Cl. A61K 8/11; A61K 8/60; A61K 8/73; A61K 8/92; A61Q 11/00

(52) U.S. Cl. CPC A61K 8/11; A61K 8/602; A61K 8/731; A61K 8/732; A61K 8/922; A61K 8/925; A61Q 11/00

(57) ABSTRACT

The present application belongs to the field of oral care materials. Specifically disclosed are a plant glycoside microcapsule, and a preparation method and application thereof. The microcapsule has a core-shell structure, and a shell of the microcapsule is a natural wax; a mass ratio of a core of the microcapsule to the shell of the microcapsule is 1:(0.3-0.7); and the natural wax includes a mixture of beeswax and carnauba in a mass ratio of 1:(0-0.2). The present application uses the natural wax as a coating material, and a plant glycoside extract is encapsulated into the microcapsule. The microcapsule is smooth in surface and compact in encapsulation.

### CROSS-REFERENCES TO RELATED APPLICATION

The present application claims the benefit of the priority of the Chinese patent application with the application No. 2024114565659.9, titled “Plant Glycoside Microcapsule, and Preparation Method and Application” filed to the China National Intellectual Property Administration on Oct. 18, 2024, the entire content of which is incorporated in this application by reference.

### TECHNICAL FIELD

The present disclosure belongs to the field of oral care materials, and specifically relates to a plant glycoside microcapsule, and a preparation method and application.

### BACKGROUND

Toothpastes are generally prepared by mixing various raw materials such as functional components, a friction agent, a humectant, a thickening agent, a foaming agent, a preservative, a sweetening agent, etc. The functional components are generally substances such as fluorides, zinc citrate, chlorhexidine, etc. At present, functional components from plants are widely used in the field of toothpastes. Glycoside compounds, formerly known as “glycosides”, are a class of biologically-active compounds widely found in plants, and their chemical structures are formed by combining sugar or sugar derivatives and non-sugars, for example, flavonoid glycoside, anthracene glycoside, anthocyanin, etc. However, most of the glycoside substances extracted from natural plants are susceptible to oxidation and may also be contraindicated with other ingredients in products, limiting their industrial application. In order to improve the stability of the glycoside substances, these natural extracts may be encapsulated in microcapsules, thereby avoiding or slowing down the oxidation and decay of their active ingredients.

Disclosed in WO2008/015186A1 is a pigment microcapsule added in a toothpaste. A water-insoluble pigment is dispersed in oil, and the oil is gelatinized to form a capsule core by adding gas phase silica; a molten wax, the oil, and titanium dioxide are mixed and added to hot suspension consisting of core materials, are stirred and mixed, and rapidly cooled to obtain the microcapsule. The microcapsule causes pigment leakage through tooth brushing friction. The pigment selected in such preparation method tends to be an oil-soluble pigment, and water-soluble glycoside substances are not suitable for this method. CN102906238A uses melamine formaldehyde or urea formaldehyde to encapsulate water-soluble active ingredients, which may be used in daily necessities such as toothpastes. However, the raw materials for the preparation of such particles are potentially toxic and unfriendly to the environment, and the use of plastic beads in cosmetics has been gradually banned. Published in WO2019/117890A1 is a method for preparing a pH sensitive microcapsule applicable to daily chemical products. Shellac and a hydroxypropyl methylcellulose derivative or acrylic ester derivative are used as a wall material. A pH sensitive microcapsule is prepared through a flow focusing technology. The microcapsule is formed by curing droplets ejected from a concentric nozzle including an external shell fluid chamber and an internal shell fluid chamber. However, common toothpastes on the market are neutral or weakly alkaline, and thus, the microcapsule is also not suitable for glycoside substance encapsulating.

Most plant-derived glycoside substances are water soluble, which makes it challenging to realize their encapsulation technologies. In particular, a toothpaste system contains a certain amount of surfactant, for example, sodium dodecyl sulfate, sodium N-lauroylsarcosinate, and cocamidopropyl betaine, so as to cause the toothpaste system to be amphiphilic to water and oil. Therefore, if it is required to cause the glycoside substances encapsulated by a microcapsule pill to not leak in an amphiphilic liquid-state environment such as the toothpaste within the shelf life or a leakage amount within an acceptable range, a coating material of the microcapsule in such environment needs to have a compact structure and an extremely-low penetration rate. Furthermore, products such as the toothpastes need to be subjected to processes such as stirring, homogenizing, etc. during production, and the microcapsule during these processes needs to be able to resist the destructive effects of friction agents, surfactants, etc. during stirring and shearing processes.

### SUMMARY

In order to overcome the above problems in the related art, one of the objectives of the present disclosure is to provide a microcapsule.

The second objective of the present disclosure is to provide a method for preparing a microcapsule.

The third objective of the present disclosure is to provide a toothpaste.

The fourth objective of the present disclosure is to provide use of the microcapsule in oral care products.

In order to achieve the above objectives, the present disclosure adopts the following technical solutions.

A first aspect of the present disclosure provides a microcapsule. The microcapsule has a core-shell structure, and a shell of the microcapsule includes a natural wax; a mass ratio of a core of the microcapsule to the shell of the microcapsule is 1:(0.3-0.7); and the natural wax includes a mixture of beeswax and carnauba in a mass ratio of 1:(0-0.2).

Preferably, the natural wax includes the mixture of the beeswax and the carnauba in the mass ratio of 1:(0.05-0.2). The present disclosure selects the mixture of the beeswax and the carnauba. The mixture has a viscosity lower than 0.3 Pa·s in a molten state, such that it can ensure that the molten natural wax continuously flows in a peristaltic tube and a nozzle, so as to coat a core material of the microcapsule by using a fluidized bed coating device.

Preferably, the core of the microcapsule includes a plant glycoside extract. Glycoside compounds are formerly known as “glycosides”; plant glycosides have a homophone in Chinese, which point to the same substance in the present disclosure, thus being collectively referred to as a plant glycoside extract below. The plant glycoside extract is an extract that uses plants as raw materials and is obtained through physical and chemical extraction and separation processes.

Preferably, the core of the microcapsule further includes a filling agent and a disintegrating agent; and a mass ratio of the plant glycoside extract, the filling agent, and the disintegrating agent is 1:(1-1.25):(0.05-0.3).

Preferably, raw materials of the plant glycoside extract are selected from at least one of scutellaria baicalensis, honeysuckle flower, coptis chinensis, licorice, rhizoma bolbostemmae, herba eupatorii, pogostemon cablin, tangerine peel, rhizoma zingiberis, chinese date, rhizoma anemarrhenae, cortex phellodendri, cortex moutan, radix rehmanniae, or radix ophiopogonis.

When the microcapsule in the present disclosure is used in a toothpaste, the microcapsule is broken due to a friction effect during tooth brushing, and water or saliva enters the microcapsule from the breakage of the microcapsule to cause the disintegrating agent in the microcapsule to disintegrate, accelerating microcapsule breakage, and thus leading to rapid releasing of the plant glycoside extract in the core of the microcapsule.

Preferably, the core of the microcapsule is a solid material.

Preferably, the disintegrating agent is selected from at least one of croscarmellose or carboxymethyl starch.

Preferably, the filling agent is selected from microcrystalline cellulose.

Preferably, a particle size of the microcapsule is 370-920 μm.

Preferably, a content of baicalin in the microcapsule is 5-6.3%.

Preferably, a coating weight gained in the microcapsule is 27.07%-57.79%.

A second aspect of the present disclosure provides a method for preparing the microcapsule provided in the first aspect of the present disclosure. The preparation method includes the following steps.

In S1: a core material of a microcapsule is prepared into a pill core.

In S2: the pill core is coated by using a natural wax, so as to prepare the microcapsule.

Preferably, S1 includes: the core material including a plant glycoside extract and a solvent are mixed, and then a pill core is prepared by using an extrusion-spheronization method; further preferably, S1 includes: the core material including the plant glycoside extract, a filling agent, and a disintegrating agent and the solvent are mixed, and then subjected to extrusion-spheronization, drying, and sieving, so as to prepare the pill core.

Preferably, the solvent is selected from water.

Preferably, a pore diameter of a screen in the extrusion-spheronization step is 0.3-0.7 mm.

Preferably, an extrusion rate of the extrusion-spheronization step is 5-10 rpm, a spheronization rate is 500-1500 rpm, and a spheronization time is 20-60 min.

Preferably, a particle size of the pill core is 0.3-0.7 mm.

Preferably, S2 includes: the natural wax is heated and melted, then the pill core is coated by using a fluidized bed coating device, and a microcapsule is prepared after cooling.

Preferably, a material temperature of the fluidized bed coating device is controlled at 25-40° C., a spray pressure is 0.1-0.2 MPa, a heating temperature of compressed air is 150-200° C., an inlet air rate is 300-800 rpm, and an exhaust air rate is 600-1200 rpm.

Preferably, a heating and melting temperature is 110-140° C.

Preferably, a centrifugal pan is disposed in the fluidized bed coating device.

Preferably, a rotary speed of the centrifugal pan is 300-500 rpm. In the present disclosure, the fluidized bed coating device with the centrifugal pan is used. The microcapsule is subjected to a combination effect of a centrifugal force, a friction force, and inlet air buoyancy. Compared to a microcapsule obtained through coating using a traditional fluidized bed, the microcapsule is smoother and more compact in surface and low in roughness, has almost no gaps or cracks, and thus can resist friction and wear during preparation of toothpaste products, thereby prolonging the storage stability of toothpastes containing the microcapsule.

A third aspect of the present disclosure provides a toothpaste, including the microcapsule provided in the first aspect of the present disclosure. The microcapsule in the present disclosure may be used in products such as toothpastes and the like. Products such as the toothpastes need to be subjected to processes such as stirring, homogenizing, etc. during production, and the microcapsule during these processes needs to resist the destructive effects of friction agents, surfactants, etc. during stirring and shearing processes. Therefore, in an aspect, for the microcapsule in the present disclosure, the plant glycoside extract may be completely encapsulated by the natural wax, such that cracks and the like cannot be present in the shell of the microcapsule; and in another aspect, the microcapsule in the present disclosure can rapidly release active ingredients when being subjected to tooth brushing friction, thereby achieving the functional effect of the plant glycoside.

Preferably, the toothpaste is a calcium carbonate toothpaste or silica toothpaste. The microcapsule in the present disclosure is smooth in surface and compact in encapsulation, can be stable for a long time in calcium carbonate and silica toothpaste substrates, and is broken and released during tooth brushing.

A fourth aspect of the present disclosure provides an application of the microcapsule provided in the first aspect of the present disclosure in oral care products.

The present disclosure has the following beneficial effects. The present disclosure uses the natural wax as a coating material, and a plant glycoside extract is encapsulated into the microcapsule. The microcapsule is smooth in surface and compact in encapsulation. Therefore, the stability of the plant glycoside extract is improved, unstable phenomena such as oxidative decomposition of glycoside components are avoided, and toothpastes containing the microcapsule have longer storage stability while the stability of the plant glycoside extract in the toothpastes is improved. Furthermore, in the present disclosure, by adjusting the component of the natural wax and component proportions, the mechanical property of the shell of the microcapsule can be changed to reduced breakage and leakage due to friction and wear of the microcapsule during paste preparation, such that most microcapsules are not broken during paste preparation, and it can also ensure that the active ingredients of the plant glycoside extract can be released during tooth brushing when the microcapsule is used in the toothpaste, thereby achieving effects of treating mouth ulcer and the like.

The preparation method in the present disclosure prepares well-encapsulated microcapsules by using the fluidized bed coating device, is fast in coating speed, high in yield, and low in cost; and the preparation method is rapid and simple, and may achieve industrial large-scale production. The preparation method used in the present disclosure may also be promoted and applied to microencapsulation of all soluble and highly diffusible active substances, achieving the purpose of isolation between the active substances and an external liquid environment and long-term stability, thus having important reference values in the field of medicine, health food, and cosmetics.

### BRIEF DESCRIPTION OF THE DRAWINGS

FIG. 1 is a test diagram of mechanical property of a natural wax mixture with different proportions in the present disclosure.

FIG. 2 is a test diagram of a surface shape of a microcapsule containing a plant glycoside extract in Embodiment 1.

FIG. 3 is a surface shape map of a calcium carbonate toothpaste in the present disclosure.

FIG. 4 is a surface shape map of a calcium carbonate toothpaste slurry in the present disclosure.

FIG. 5 is a test diagram of a surface shape of a microcapsule containing a plant glycoside extract in Embodiment 1.

FIG. 6 is a surface shape map of a silica toothpaste in the present disclosure.

FIG. 7 is a surface shape map of a silica toothpaste slurry in the present disclosure.

### DETAILED DESCRIPTION OF THE EMBODIMENTS

Specific implementations of the present disclosure are further described in detail below with reference to the drawings and examples, but the implementations and protection of the present disclosure are not limited thereto. It is to be noted that the following processes, if any, which are not described in particular detail, may be realized or understood by those skilled in the art with reference to the related art. Reagents or instruments used of which the manufacturers are not indicated are conventional products that are commercially available.

The present disclosure studies the performance of natural waxes of beeswax (W1) and carnauba (W3) in different mass percentage ratios, that is, a natural wax mixture (a mixture of the W1 and the W3, recorded as W1-5% W3) containing the W3 with the mass percentage ratio of 5%, a natural wax mixture (recorded as W1-10% W3) containing the W3 with the mass percentage ratio of 10%, a natural wax mixture (recorded as W1-15% W3) containing the W3 with the mass percentage ratio of 15%, a natural wax mixture (recorded as W1-20% W3) containing the W3 with the mass percentage ratio of 20%, a natural wax mixture (recorded as W1-30% W3) containing the W3 with the mass percentage ratio of 30%, a natural wax mixture (recorded as W1-40% W3) containing the W3 with the mass percentage ratio of 40%, and a natural wax mixture (recorded as W1-50% W3) containing the W3 with the mass percentage ratio of 50% are respectively prepared, and then the following performance of these natural wax mixtures is tested.

### (1) Mechanical Property

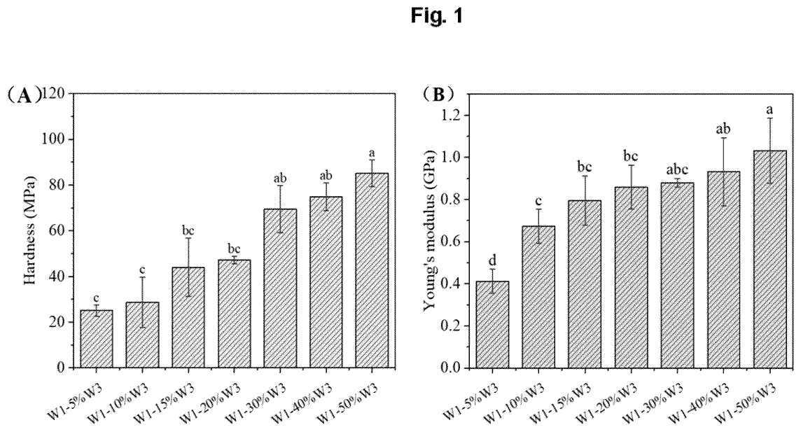

The hardness and Young's modulus of the above natural wax mixtures are tested, and specific test results are shown in FIG. 1. (A) in FIG. 1 and (B) in FIG. 1 respectively are test diagrams of the hardness and the Young's modulus. From FIG. 1, it may be learned that, when the mass percentage ratio of the W3 in the natural wax is 5%, the hardness and Young's modulus of the W1-5% W3 respectively are 25.06 MPa and 0.41 GPa, and as the content of the W3 increases, the hardness and the Young's modulus increase accordingly. In the present disclosure, a shell of a microcapsule needs to be able to be stably stored in a toothpaste and can release a core material during tooth brushing, through comprehensive consideration, when the natural wax is a mixture of the beeswax and the carnauba in a mass ratio of 1:(0.05-0.2), a use requirement of the microcapsule may be met.

### (2) Weight Loss Ratio

The above natural wax mixtures respectively are stored at 45° C. for 90 days in calcium carbonate and silica toothpastes in an accelerated manner, then weight loss ratios of the natural wax mixtures are tested, and specific test results are as follows: after the above natural wax mixtures are stored for 90 days in the calcium carbonate toothpaste, the weight loss ratios are 7.37-11.19%, with the maximum weight loss ratio being W1-15% W3 and the minimum weight loss ratio being W1-10% W2. The weight loss ratios for 90 days of accelerated storage in the silica toothpaste are all lower than 0.5%. Then surface shapes of the above natural wax mixtures after 90 days of storage are respectively observed by using a scanning electron microscope. It may be learned that, the above natural wax mixtures hardly corrode in the silica and calcium carbonate toothpastes, are good in state, and may stably encapsulate a plant glycoside extract.

Raw materials and test methods used in the embodiments and comparative examples of the present disclosure are as follows.

The plant glycoside extract is an extract of scutellaria baicalensis and honeysuckle flower, and baicalin is used as a marker for testing during test processes of the embodiments.

A method for preparing the plant glycoside extract includes: scutellaria baicalensis root and honeysuckle flower medicinal materials are ground into powder, then mixed with ethanol, and subjected to reflux extraction through heating at 60-80° C., and the plant glycoside extract is prepared through filtration and drying.

A method for testing the baicalin includes: a pill core or microcapsule is ground by using a mortar, then 25 mg is accurately weighed, and the mass is recorded as M1; 20 mL of an ethanol-monophosphate solution (in the ethanol-monophosphate solution, a volume ratio of anhydrous ethanol, phosphoric acid, and water is 50:6:44) is added, vortex oscillation is performed for 2 min, ultrasound extraction is performed for 40 min in a 25° C. water bath, centrifugation is performed for 10 min at 5000 rpm, and supernatant is made up to 25 mL by using the ethanol-phosphoric acid aqueous solution (in the ethanol-phosphoric acid aqueous solution, the volume ratio of the anhydrous ethanol, the phosphoric acid, and the water is 50:6:44). A 0.22 μm filter membrane is used for filtration, and filtrate is taken as a sample solution. 10 μL is accurately sucked with a high performance liquid chromatography, and chromatographic conditions include: C18 chromatographic column 250 mm×4.6 mm (a particle size of 5 μm), and column temperature 30° C.; mobile phase: acetonitrile and 0.1% phosphoric acid solution, gradient elution (0-2 min: 13% acetonitrile, 87% phosphoric acid solution; 18-25 min: 30% acetonitrile, 70% phosphoric acid solution; 28-30 min: 13% acetonitrile, 87% phosphoric acid solution); flow rate: 1.0 mL/min; detection wavelength: 277 nm. The content M2 of the baicalin in the pill core or microcapsule is obtained through testing and calculation. Drug-loading rate=M2/M1×100%, in the formula, M2 is the content of the baicalin in the pill core or microcapsule, and M1 is the mass of the pill core or microcapsule.

Method for testing actual coating weight gained: actual weight gained (%)=(drug-loading rate of pill core-drug-loading rate in microcapsule)/drug-loading rate in microcapsule×100%.

Calcium carbonate toothpaste substrate formula: 30% of a humectant, 40% of calcium carbonate, 5% of hydrated silica, 2% of a surfactant, 0.8% of a thickening agent, 0.2% of a sweetening agent, 1.5% of a fragrance agent, and 0.5% of a preservative, with the balance being water. A calcium carbonate toothpaste substrate is obtained through mixing, stirring, homogenizing, and vacuum degassing in a paste preparation machine.

Silica toothpaste substrate formula: 45% of a humectant, 25% of hydrated silica, 2% of a surfactant, 0.8% of a thickening agent, 0.2% of a sweetening agent, 1.5% of a fragrance agent, and 0.5% of a preservative, with the balance being water. A silica toothpaste substrate is obtained through mixing, stirring, homogenizing, and vacuum degassing in the paste preparation machine.

### Embodiment 1

This example provided a microcapsule containing a plant glycoside extract. The microcapsule was prepared by using the following preparation method, and specific steps were as follows.

The plant glycoside extract, microcrystalline cellulose, and croscarmellose were uniformly mixed according to a mass ratio of 1:1:0.05, and moisture of 50% of the mass of dry materials was added, so as to prepare into a soft material. A perforated plate with a pore diameter of 300 μm was selected, a screw extrusion rate was 5 rpm, a spheronization rate was 1500 rpm, a spheronization time was 20 min, a spherical particle was prepared, and the spherical particle was dried at 35° C. and sieved, so as to obtain a pill core.

Beeswax and carnauba were hot-melted and mixed according to a mass ratio of 1:0.05 as a coating solution, and a mass ratio of the pill core to a coating material was 1:0.5. A coating was hot-melted by using a tangential fluidized bed, a material temperature of a fluidized bed coating machine was controlled at 30° C., an inlet air rotary speed of the coating machine was 600 rpm, an exhaust air rotary speed was 1200 rpm, a rotary speed of a centrifugal pan was 300 rpm, a compressed air temperature was set to 170° C., the coating solution was heated to 130° C., an atomization pressure was 0.1 MPa, and sieving was performed after coating was completed, so as to obtain the microcapsule containing the plant glycoside extract in this example.

Through detection, a yield of the prepared microcapsule containing the plant glycoside extract was 87.46%, an average particle size of the microcapsule was 376.65 μm, a drug-loading rate of baicalin was 5.62%, and a coating weight gained was 35.29%.

### Embodiment 2

This example provided a microcapsule containing a plant glycoside extract. The microcapsule was prepared by using the following preparation method, and specific steps were as follows.

The plant glycoside extract, microcrystalline cellulose, and carboxymethyl starch were uniformly mixed according to a mass ratio of 1:1.25:0.25, and moisture of 50% of the mass of dry materials was added, so as to prepare into a soft material. A perforated plate with a pore diameter of 400 μm was selected, a screw extrusion rate was 5 rpm, a spheronization rate was 800 rpm, a spheronization time was 30 min, and a spherical particle was prepared, dried at 35° C. and sieved, so as to obtain a pill core.

Beeswax and carnauba were hot-melted and mixed according to a mass ratio of 1:0.05 as a coating solution, and a mass ratio of the pill core to the coating solution was 1:0.5. A coating was hot-melted by using a tangential fluidized bed, a material temperature of a fluidized bed coating machine was controlled at 30° C., an inlet air rotary speed of the coating machine was 600 rpm, an exhaust air rotary speed was 1200 rpm, a rotary speed of a centrifugal pan was 400 rpm, a compressed air temperature was set to 170° C., the coating solution was heated to 130° C., an atomization pressure was 0.1 MPa. Sieving was performed after coating was completed, so as to obtain the microcapsule containing the plant glycoside extract in this example.

Through detection, a yield of the prepared microcapsule was 83.92%, an average particle size of the microcapsule was 483.53 μm, a drug-loading rate of baicalin was 5.44%, and a coating weight gained was 37.00%.

### Embodiment 3

This example provided a microcapsule containing a plant glycoside extract. The microcapsule was prepared by using the following preparation method, and specific steps were as follows.

The plant glycoside extract, microcrystalline cellulose, and croscarmellose were uniformly mixed according to a mass ratio of 1:1.25:0.25, and moisture of 40% of the mass of dry materials was added, so as to prepare into a soft material. A perforated plate with a pore diameter of 500 μm was selected, a screw extrusion rate was 8 rpm, a spheronization rate was 500 rpm, a spheronization time was 60 min, and a spherical particle was prepared, dried at 35° C. and sieved, so as to obtain a pill core.

Beeswax and carnauba were hot-melted and mixed according to a mass ratio of 1:0.05 as a coating solution, and a mass ratio of the pill core to the coating solution was 1:0.5. A coating was hot-melted by using a tangential fluidized bed, a material temperature of a fluidized bed coating machine was controlled at 30° C., an inlet air rotary speed of the coating machine was 600 rpm, an exhaust air rotary speed was 1200 rpm, a rotary speed of a centrifugal pan was 300 rpm, a compressed air temperature was set to 170° C., the coating solution was heated to 130° C., an atomization pressure was 0.1 MPa. Sieving was performed after coating was completed, so as to obtain the microcapsule containing the plant glycoside extract in this example.

Through detection, a yield of the prepared microcapsule was 81.56%, an average particle size of the microcapsule was 610.54 μm, a drug-loading rate of baicalin was 5.55%, and a coating weight gained was 37.38%.

### Embodiment 4

This example provided a microcapsule containing a plant glycoside extract. The microcapsule was prepared by using the following preparation method, and specific steps were as follows.

The plant glycoside extract, microcrystalline cellulose, and croscarmellose were uniformly mixed according to a mass ratio of 1:1.25:0.3, and moisture of 40% of the mass of dry materials was added, so as to prepare into a soft material. A perforated plate with a pore diameter of 600 μm was selected, a screw extrusion rate was 8 rpm, a spheronization rate was 800 rpm, a spheronization time was 30 min, and a spherical particle was prepared, dried at 35° C. and sieved, so as to obtain a pill core.

Beeswax and carnauba were hot-melted and mixed according to a mass ratio of 1:0.05 as a coating solution, and a mass ratio of the pill core to the coating solution was 1:0.5. A coating was hot-melted by using a tangential fluidized bed, a material temperature of a fluidized bed coating machine was controlled at 30° C., an inlet air rotary speed of the coating machine was 300 rpm, an exhaust air rotary speed was 600 rpm, a rotary speed of a centrifugal pan was 300 rpm, a compressed air temperature was set to 170° C., the coating solution was heated to 130° C., an atomization pressure was 0.1 MPa. Sieving was performed after coating was completed, so as to obtain the microcapsule containing the plant glycoside extract in this example.

Through detection, a yield of the prepared microcapsule was 78.43%, an average particle size of the microcapsule was 698.24 μm, a drug-loading rate of baicalin was 5.55%, and a coating weight gained was 37.40%.

### Embodiment 5

This example provided a microcapsule containing a plant glycoside extract. The microcapsule was prepared by using the following preparation method, and specific steps were as follows.

The plant glycoside extract, microcrystalline cellulose, and croscarmellose were uniformly mixed according to a mass ratio of 1:1.25:0.25, and moisture of 40% of the mass of dry materials was added, so as to prepare into a soft material. A perforated plate with a pore diameter of 700 μm was selected, a screw extrusion rate was 10 rpm, a spheronization rate was 800 rpm, a spheronization time was 30 min, and a spherical particle was prepared, dried at 35° C. and sieved, so as to obtain a pill core.

Beeswax and carnauba were hot-melted and mixed according to a mass ratio of 1:0.05 as a coating solution, and a mass ratio of the pill core to the coating solution was 1:0.5. A coating was hot-melted by using a tangential fluidized bed, a material temperature of a fluidized bed coating machine was controlled at 30° C., an inlet air rotary speed of the coating machine was 600 rpm, an exhaust air rotary speed was 1200 rpm, a rotary speed of a centrifugal pan was 300 rpm, a compressed air temperature was set to 170° C., the coating solution was heated to 130° C., an atomization pressure was 0.15 MPa. Sieving was performed after coating was completed, so as to obtain the microcapsule containing the plant glycoside extract in this example.

Through detection, a yield of the prepared microcapsule was 72.45%, an average particle size of the microcapsule was 806.31 μm, a drug-loading rate of baicalin was 5.39%, and a coating weight gained was 39.42%.

### Embodiment 6

This example provided a microcapsule containing a plant glycoside extract. The microcapsule was prepared by using the following preparation method, and specific steps were as follows.

The plant glycoside extract, microcrystalline cellulose, and croscarmellose were uniformly mixed according to a mass ratio of 1:1.25:0.25, and moisture of 40% of the mass of dry materials was added, so as to prepare into a soft material. A perforated plate with a pore diameter of 700 μm was selected, a screw extrusion rate was 8 rpm, a spheronization rate was 800 rpm, a spheronization time was 30 min, and a spherical particle was prepared, dried at 35° C. and sieved, so as to obtain a pill core.

Beeswax and carnauba were hot-melted and mixed according to a mass ratio of 1:0 as a coating solution, and a mass ratio of the pill core to the coating solution was 1:0.5. A coating was hot-melted by using a tangential fluidized bed, a material temperature of a fluidized bed coating machine was controlled at 30° C., an inlet air rotary speed of the coating machine was 600 rpm, an exhaust air rotary speed was 1200 rpm, a rotary speed of a centrifugal pan was 300 rpm, a compressed air temperature was set to 150° C., the coating solution was heated to 110° C., an atomization pressure was 0.2 MPa. Sieving was performed after coating was completed, so as to obtain the microcapsule containing the plant glycoside extract in this example.

Through detection, a yield of the prepared microcapsule was 45.78%, an average particle size of the microcapsule was 816.43 μm, a drug-loading rate of baicalin was 5.65%, and a coating weight gained was 34.65%.

### Embodiment 7

This example provided a microcapsule containing a plant glycoside extract. The microcapsule was prepared by using the following preparation method, and specific steps were as follows.

The plant glycoside extract, microcrystalline cellulose, and croscarmellose were uniformly mixed according to a mass ratio of 1:1.25:0.25, and moisture of 40% of the mass of dry materials was added, so as to prepare into a soft material. A perforated plate with a pore diameter of 700 μm was selected, a screw extrusion rate was 8 rpm, a spheronization rate was 800 rpm, a spheronization time was 30 min, and a spherical particle was prepared, dried at 35° C. and sieved, so as to obtain a pill core.

Beeswax and carnauba were hot-melted and mixed according to a mass ratio of 1:0.11 as a coating solution, and a mass ratio of the pill core to the coating solution was 1:0.5. A coating was hot-melted by using a tangential fluidized bed, a material temperature of a fluidized bed coating machine was controlled at 30° C., an inlet air rotary speed of the coating machine was 600 rpm, an exhaust air rotary speed was 1200 rpm, a rotary speed of a centrifugal pan was 300 rpm, a compressed air temperature was set to 170° C., the coating solution was heated to 130° C., an atomization pressure was 0.1 MPa. Sieving was performed after coating was completed, so as to obtain the microcapsule containing the plant glycoside extract in this example.

Through detection, a yield of the prepared microcapsule was 74.53%, an average particle size of the microcapsule was 818.34 μm, a drug-loading rate of baicalin was 5.62%, and a coating weight gained was 34.56%.

### Embodiment 8

This example provided a microcapsule containing a plant glycoside extract. The microcapsule was prepared by using the following preparation method, and specific steps were as follows.

The plant glycoside extract, microcrystalline cellulose, and croscarmellose were uniformly mixed according to a mass ratio of 1:1.25:0.25, and moisture of 40% of the mass of dry materials was added, so as to prepare into a soft material. A perforated plate with a pore diameter of 700 μm was selected, a screw extrusion rate was 8 rpm, a spheronization rate was 800 rpm, a spheronization time was 30 min, and a spherical particle was prepared, dried at 35° C. and sieved, so as to obtain a pill core.

Beeswax and carnauba were hot-melted and mixed according to a mass ratio of 1:0.18 as a coating solution, and a mass ratio of the pill core to the coating solution was 1:0.5. A coating was hot-melted by using a tangential fluidized bed, a material temperature of a fluidized bed coating machine was controlled at 30° C., an inlet air rotary speed of the coating machine was 600 rpm, an exhaust air rotary speed was 1200 rpm, a rotary speed of a centrifugal pan was 300 rpm, a compressed air temperature was set to 170° C., the coating solution was heated to 130° C., an atomization pressure was 0.1 MPa. Sieving was performed after coating was completed, so as to obtain the microcapsule containing the plant glycoside extract in this example.

Through detection, a yield of the prepared microcapsule was 75.89%, an average particle size of the microcapsule was 806.56 μm, a drug-loading rate of baicalin was 5.69%, and a coating weight gained was 33.52%.

### Embodiment 9

This example provided a microcapsule containing a plant glycoside extract. The microcapsule was prepared by using the following preparation method, and specific steps were as follows.

The plant glycoside extract, microcrystalline cellulose, and croscarmellose were uniformly mixed according to a mass ratio of 1:1.25:0.25, and moisture of 40% of the mass of dry materials was added, so as to prepare into a soft material. A perforated plate with a pore diameter of 700 μm was selected, a screw extrusion rate was 8 rpm, a spheronization rate was 800 rpm, a spheronization time was 30 min, and a spherical particle was prepared, dried at 35° C. and sieved, so as to obtain a pill core.

Beeswax and carnauba were hot-melted and mixed according to a mass ratio of 1:0.25 as a coating solution, and a mass ratio of the pill core to the coating solution was 1:0.5. A coating was hot-melted by using a tangential fluidized bed, a material temperature of a fluidized bed coating machine was controlled at 30° C., an inlet air rotary speed of the coating machine was 600 rpm, an exhaust air rotary speed was 1200 rpm, a rotary speed of a centrifugal pan was 300 rpm, a compressed air temperature was set to 190° C., the coating solution was heated to 140° C., an atomization pressure was 0.1 MPa. Sieving was performed after coating was completed, so as to obtain the microcapsule containing the plant glycoside extract in this example.

Through detection, a yield of the prepared microcapsule was 83.52%, an average particle size of the microcapsule was 833.53 μm, a drug-loading rate of baicalin was 5.47%, and a coating weight gained was 39.04%.

### Embodiment 10

This example provided a microcapsule containing a plant glycoside extract. The microcapsule was prepared by using the following preparation method, and specific steps were as follows.

The plant glycoside extract, microcrystalline cellulose, and croscarmellose were uniformly mixed according to a mass ratio of 1:1.25:0.25, and moisture of 40% of the mass of dry materials was added, so as to prepare into a soft material. A perforated plate with a pore diameter of 700 μm was selected, a screw extrusion rate was 8 rpm, a spheronization rate was 800 rpm, a spheronization time was 25 min, and a spherical particle was prepared, dried at 35° C. and sieved, so as to obtain a pill core.

Beeswax and carnauba were hot-melted and mixed according to a mass ratio of 1:0.05 as a coating solution, and a mass ratio of the pill core to the coating solution was 1:0.3. A coating was hot-melted by using a tangential fluidized bed, a material temperature of a fluidized bed coating machine was controlled at 30° C., an inlet air rotary speed of the coating machine was 600 rpm, an exhaust air rotary speed was 1200 rpm, a rotary speed of a centrifugal pan was 300 rpm, a compressed air temperature was set to 170° C., the coating solution was heated to 130° C., an atomization pressure was 0.1 MPa. Sieving was performed after coating was completed, so as to obtain the microcapsule containing the plant glycoside extract in this example.

Through detection, a yield of the prepared microcapsule was 91.98%, an average particle size of the microcapsule was 776.65 μm, a drug-loading rate of baicalin was 6.22%, and a coating weight gained was 27.07%.

### Embodiment 11

This example provided a microcapsule containing a plant glycoside extract. The microcapsule was prepared by using the following preparation method, and specific steps were as follows.

The plant glycoside extract, microcrystalline cellulose, and croscarmellose were uniformly mixed according to a mass ratio of 1:1.25:0.25, and moisture of 40% of the mass of dry materials was added, so as to prepare into a soft material. A perforated plate with a pore diameter of 700 μm was selected, a screw extrusion rate was 8 rpm, a spheronization rate was 800 rpm, a spheronization time was 30 min, and a spherical particle was prepared, dried at 35° C. and sieved, so as to obtain a pill core.

Beeswax and carnauba were hot-melted and mixed according to a mass ratio of 1:0.05 as a coating solution, and a mass ratio of the pill core to the coating solution was 1:0.4. A coating was hot-melted by using a tangential fluidized bed, a material temperature of a fluidized bed coating machine was controlled at 30° C., an inlet air rotary speed of the coating machine was 600 rpm, an exhaust air rotary speed was 1200 rpm, a rotary speed of a centrifugal pan was 300 rpm, a compressed air temperature was set to 170° C., the coating solution was heated to 130° C., an atomization pressure was 0.1 MPa. Sieving was performed after coating was completed, so as to obtain the microcapsule containing the plant glycoside extract in this example.

Through detection, a yield of the prepared microcapsule was 81.46%, an average particle size of the microcapsule was 769.32 μm, a drug-loading rate of baicalin was 5.92%, and a coating weight gained was 33.60%.

### Embodiment 12

This example provided a microcapsule containing a plant glycoside extract. The microcapsule was prepared by using the following preparation method, and specific steps were as follows.

The plant glycoside extract, microcrystalline cellulose, and croscarmellose were uniformly mixed according to a mass ratio of 1:1.25:0.25, and moisture of 50% of the mass of dry materials was added, so as to prepare into a soft material. A perforated plate with a pore diameter of 700 μm was selected, a screw extrusion rate was 8 rpm, a spheronization rate was 800 rpm, a spheronization time was 30 min, and a spherical particle was prepared, dried at 35° C. and sieved, so as to obtain a pill core.

Beeswax and carnauba were hot-melted and mixed according to a mass ratio of 1:0.05 as a coating solution, and a mass ratio of the pill core to the coating solution was 1:0.6. A coating was hot-melted by using a tangential fluidized bed, a material temperature of a fluidized bed coating machine was controlled at 30° C., an inlet air rotary speed of the coating machine was 600 rpm, an exhaust air rotary speed was 1200 rpm, a rotary speed of a centrifugal pan was 300 rpm, a compressed air temperature was set to 170° C., the coating solution was heated to 130° C., an atomization pressure was 0.1 MPa. Sieving was performed after coating was completed, so as to obtain the microcapsule containing the plant glycoside extract in this example.

Through detection, a yield of the prepared microcapsule was 70.46%, an average particle size of the microcapsule was 845.98 μm, a drug-loading rate of baicalin was 5.25%, and a coating weight gained was 50.48%.

### Embodiment 13

This example provided a microcapsule containing a plant glycoside extract. The microcapsule was prepared by using the following preparation method, and specific steps were as follows.

The plant glycoside extract, microcrystalline cellulose, and croscarmellose were uniformly mixed according to a mass ratio of 1:1.25:0.25, and moisture of 50% of the mass of dry materials was added, so as to prepare into a soft material. A perforated plate with a pore diameter of 700 μm was selected, a screw extrusion rate was 8 rpm, a spheronization rate was 800 rpm, a spheronization time was 30 min, and a spherical particle was prepared, dried at 35° C. and sieved, so as to obtain a pill core.

Beeswax and carnauba were hot-melted and mixed according to a mass ratio of 1:0.05 as a coating solution, and a mass ratio of the pill core to the coating solution was 1:0.7. A coating was hot-melted by using a tangential fluidized bed, a material temperature of a fluidized bed coating machine was controlled at 30° C., an inlet air rotary speed of the coating machine was 600 rpm, an exhaust air rotary speed was 1200 rpm, a rotary speed of a centrifugal pan was 300 rpm, a compressed air temperature was set to 170° C., the coating solution was heated to 130° C., an atomization pressure was 0.1 MPa. Sieving was performed after coating was completed, so as to obtain the microcapsule containing the plant glycoside extract in this example.

Through detection, a yield of the prepared microcapsule was 66.41%, an average particle size of the microcapsule was 916.24 μm, a drug-loading rate of baicalin was 5.01%, and a coating weight gained was 57.79%.

### Comparative Example 1

This example provided a microcapsule containing a plant glycoside extract. The microcapsule was prepared by using the following preparation method, and specific steps were as follows.

The plant glycoside extract, microcrystalline cellulose, and croscarmellose were uniformly mixed according to a mass ratio of 1:1:0.05, and moisture of 50% of the mass of dry materials was added, so as to prepare into a soft material. A perforated plate with a pore diameter of 300 μm was selected, a screw extrusion rate was 5 rpm, a spheronization rate was 1500 rpm, a spheronization time was 20 min, and a spherical particle was prepared, dried at 35° C. and sieved, so as to obtain a pill core.

A candelilla wax was hot-melted and mixed as a coating solution, and a mass ratio of the pill core to a coating material was 1:0.5. A coating was hot-melted by using a tangential fluidized bed, a material temperature of a fluidized bed coating machine was controlled at 30° C., an inlet air rotary speed of the coating machine was 600 rpm, an exhaust air rotary speed was 1200 rpm, a rotary speed of a centrifugal pan was 300 rpm, a compressed air temperature was set to 180° C., the coating solution was heated to 140° C., an atomization pressure was 0.1 MPa. Sieving was performed after coating was completed, so as to obtain the microcapsule containing the plant glycoside extract in this example.

Through detection, a yield of the prepared microcapsule was 65.56%, an average particle size of the microcapsule was 356.53 μm, a drug-loading rate of baicalin was 5.22%, and a coating weight gained was 30.29%.

### Comparative Example 2

This example provided a microcapsule containing a plant glycoside extract. The microcapsule was prepared by using the following preparation method, and specific steps were as follows.
(1) Shellac particles were added to a NaOH solution, stirred, and heated to 70° C. for dissolution, the NaOH solution was added dropwise to maintain pH=7.3 during dissolution, and a shellac solution with a mass concentration of 35% was prepared; octenyl succinate starch (a molecular weight being 1.2×10⁴ g/mol, and a degree of substitution being 0.0105) was added to water, the mixture was heated at 95° C. for 30 min and gelatinized into an octenyl succinate starch solution with a mass concentration of 35%, and pH=7.4 was regulated; and a baicalin extract was added to the water and stirred to dissolve, a plant glycoside solution with a mass concentration of 35% was prepared, and pH=7.0 was regulated with the NaOH solution. According to a mass ratio of the shellac, the octenyl succinate starch, and the baicalin extract being 1:0.25:0.42, 20 g of the shellac solution, 5 g of the octenyl succinate starch solution, and 8.33 g of a baicalin extract solution were taken, mixed, and stirred uniformly.
(2) 30 g of the mixed solution was taken and dropwise added to 300 g of corn oil at a flow rate of 3.3 mL/min (a mass ratio of the mixed solution to the plant oil being 1:10), and stirring was performed at a rotary speed of 400 rpm. After 30 min of stirring, 6.30 g of CaCl₂) powder was slowly added according to a mass ratio of CaCl₂) to the shellac being 1:1, and the reaction was continuously stirred at 25° C. for 4 h, and was allowed to stand for 12 h. The reaction system was centrifuged for 5 min at 4000 rpm, and then upper oil was removed. Hexane was added, centrifugation was performed for 5 min at 4000 r/min, and washing was repeatedly performed for 3 times. Then, the microcapsule was washed with distilled water, subjected to suction filtration, and dried for 12 h at 35° C., so as to obtain the microcapsule containing the plant glycoside extract in this example, and an average particle size of the microcapsule was 136 μm.

### Comparative Example 3

This example provided a microcapsule containing a plant glycoside extract. The microcapsule was prepared by using the following preparation method, and specific steps were as follows.
(1) Shellac particles were added to a NaOH solution, stirred, and heated to 70° C. for dissolution, the NaOH solution was added dropwise to maintain pH=7.4 during dissolution, and a shellac solution with a mass concentration of 35% was prepared; octenyl succinate starch (a molecular weight being 1.2×10⁴ g/mol, and a degree of substitution being 0.0105) was added to water, the mixture was heated at 95° C. for 30 min and gelatinized into an octenyl succinate starch solution with a mass concentration of 35%, and pH=7.0 was regulated; and a baicalin extract was added to the water and stirred to dissolve, a plant glycoside solution with a mass concentration of 35% was prepared, and pH=7.4 was regulated with the NaOH solution. According to a mass ratio of the shellac, the octenyl succinate starch, and the plant glycoside being 1:1:2, 10 g of the shellac solution, 10 g of the octenyl succinate starch solution, and 20 g of the plant glycoside solution were taken, mixed, and stirred uniformly.
(2) 30 g of the mixed solution was taken and dropwise added to 300 g of soybean oil at a flow rate of 3.3 mL/min (a mass ratio of the mixed solution to the plant oil being 1:10), and stirring was performed at a rotary speed of 400 rpm. After 30 min of stirring, 2.63 g of CaCl₂) powder was slowly added according to a mass ratio of CaCl₂) to the shellac being 1:1, and the reaction was continuously stirred at 25° C. for 4 h, and was allowed to stand for 12 h. The reaction system was centrifuged for 5 min at 4000 rpm, and then upper oil was removed. Hexane was added, centrifugation was performed for 5 min at 4000 r/min, and washing was repeatedly performed for 3 times. Then, the microcapsule was washed with distilled water, subjected to suction filtration, and dried for 12 h at 35° C., so as to obtain the microcapsule containing the plant glycoside extract in this example. An average particle size of the microcapsule was 109 μm.

### Performance Test:

### (1) Surface Shape Test

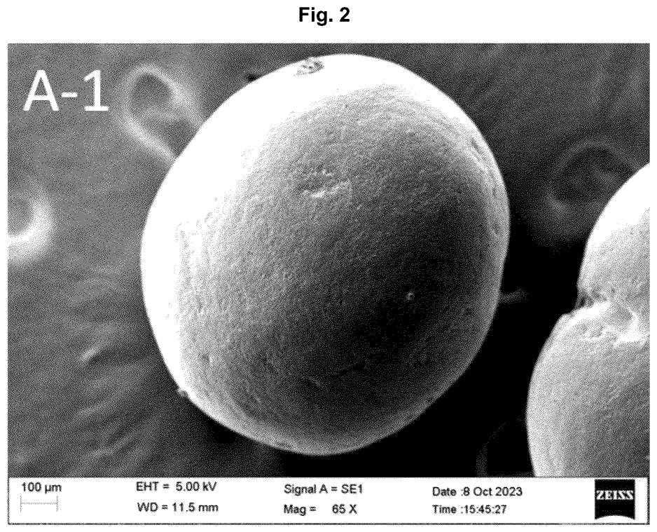

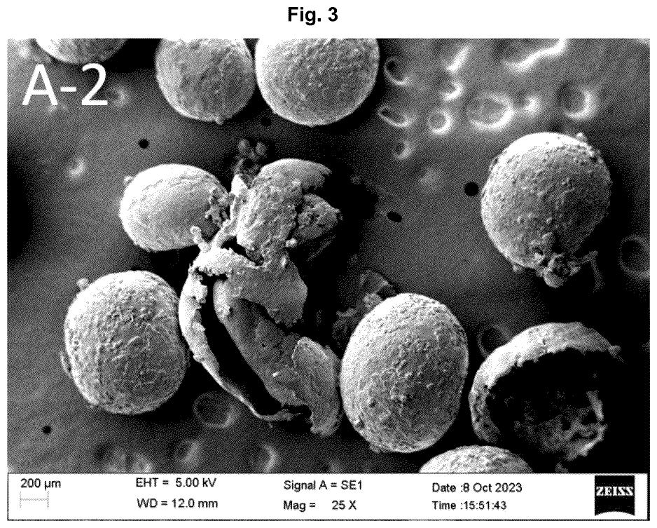

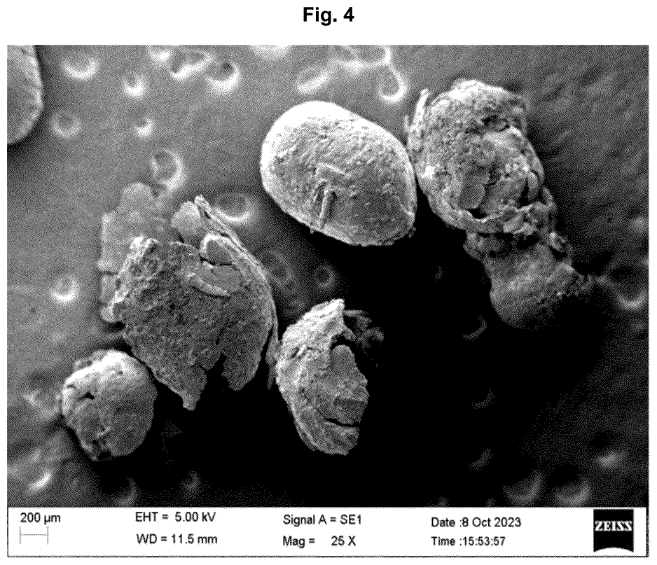

1 g of the microcapsule containing the plant glycoside extract prepared in Embodiment 1 and 39 g of a calcium carbonate toothpaste substrate were stirred for 1 min at a rotary speed of 50 rpm for uniform mixing, so as to prepare a calcium carbonate toothpaste with the microcapsule containing the plant glycoside extract; and 1 g of the calcium carbonate toothpaste with the microcapsule containing the plant glycoside extract was taken, tooth brushing was simulated for 3 min on resin dentures, a consistent force was maintained during the brushing of the toothpaste, the resin dentures were washed with water after brushing, and all toothpaste slurry was collected. Then, surface shape maps of the microcapsule containing the plant glycoside extract prepared in Embodiment 1, the calcium carbonate toothpaste, and the calcium carbonate toothpaste slurry were tested by using a scanning electron microscope. Specific test results were respectively shown in FIG. 2, FIG. 3, and FIG. 4.

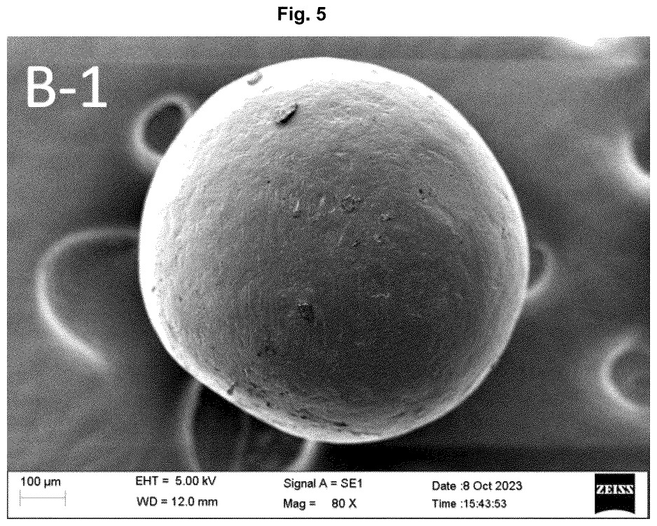

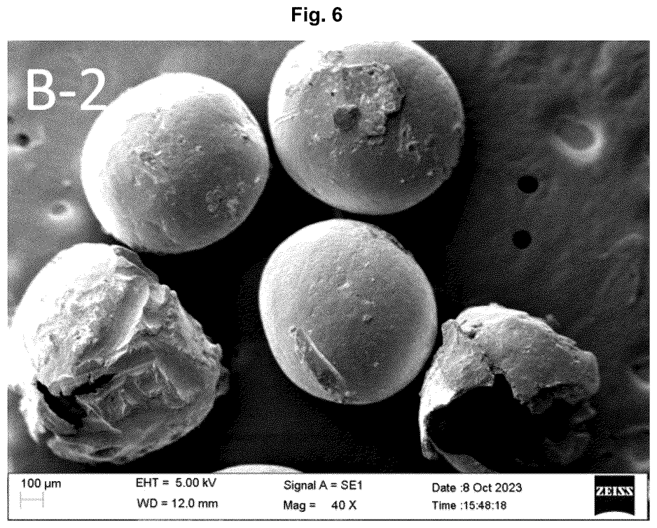

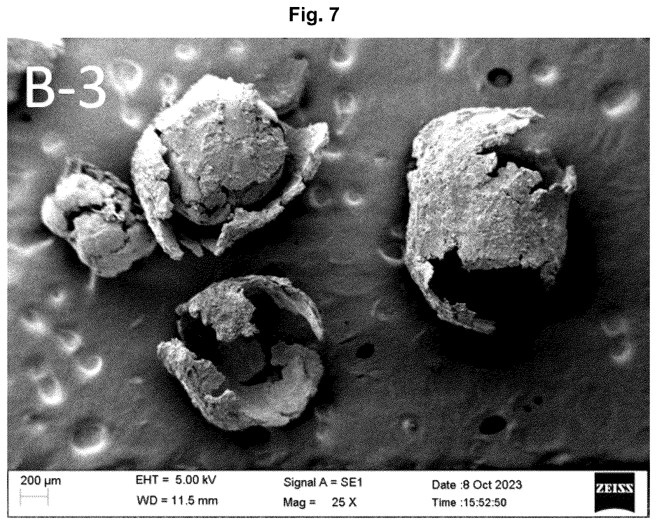

1 g of the microcapsule containing the plant glycoside extract prepared in Embodiment 1 and 39 g of a silica toothpaste substrate were stirred for 1 min at a rotary speed of 50 rpm for uniform mixing, so as to prepare a silica toothpaste with the microcapsule containing the plant glycoside extract; and 1 g of the silica toothpaste with the microcapsule containing the plant glycoside extract was taken, tooth brushing was simulated for 3 min on resin dentures, a consistent force was maintained during the brushing of the toothpaste, the resin dentures were washed with water after brushing, and all toothpaste slurry was collected. Then, surface shape maps of the microcapsule containing the plant glycoside extract prepared in Embodiment 1, the silica toothpaste, and the silica toothpaste slurry were tested by using a scanning electron microscope. Specific test results were respectively shown in FIG. 5, FIG. 6, and FIG. 7.

From FIG. 2-FIG. 7, it may be learned that, the microcapsule containing the plant glycoside extract prepared in the present disclosure was of a near-spherical structure and relatively-smooth in surface, and the plant glycoside extract was encapsulated in the microcapsule. When the microcapsule and the toothpaste substrate were uniformly mixed and stirred, the friction agent silica particles or calcium carbonate particles had an abrasive action on the surfaces of the microcapsule, leading to reduction in the protection of the coating layer of a portion of the microcapsule to the pill core; liquid in the toothpaste entered the inside through the breakage of the microcapsule or a position in which original encapsulation was not tight, leading to water absorption, expansion, and disintegration of the disintegrating agent in the pill core, thus causing the portion of the microcapsule to break; however, most of the microcapsules were not broken and were stable, indicating the stability of the microcapsule structure in the present disclosure. Furthermore, through the mechanical action of simulated tooth brushing, most of the microcapsules in the toothpaste were broken, the pill cores disintegrated and dissolved, and only an “empty shell” formed by a wax film coating material was left. By comparing a surface of the wax film coating layer before and after tooth brushing, it might be obviously found that tooth brushing friction caused the surface of the wall material to be rougher. After tooth brushing friction, microcapsule breakage in the calcium carbonate toothpaste was more serious than that in the silica toothpaste, which was caused by a difference in the friction agent in the toothpaste system.

### (2) Leakage Rate, Release Rate, and Release Increment Tests for Baicalin

The microcapsules containing the plant glycoside extracts in Embodiments 1-13 and Comparative example 1 were respectively mixed with the calcium carbonate toothpaste substrate and the silica toothpaste substrate, so as to prepare the calcium carbonate toothpaste with the microcapsule containing the plant glycoside extract and the silica toothpaste with the microcapsule containing the plant glycoside extract. A specific preparation method included as follows.
(1) Preparation of the calcium carbonate toothpaste with the microcapsule containing the plant glycoside extract: 1 g of each of the microcapsules containing the plant glycoside extracts prepared in Embodiments 1-13 and Comparative example 1 were respectively taken and stirred with 39 g of the calcium carbonate toothpaste substrate for 1 min at a rotary speed of 50 rpm for well mixing, so as to prepare the calcium carbonate toothpaste with the microcapsule containing the plant glycoside extract.
(2) Preparation of the silica toothpaste with the microcapsule containing the plant glycoside extract: 1 g of each of the microcapsules containing the plant glycoside extracts prepared in Embodiments 1-13 and Comparative example 1 were respectively taken and stirred with 39 g of the silica toothpaste substrate for 1 min at a rotary speed of 50 rpm for well mixing, so as to prepare the silica toothpaste with the microcapsule containing the plant glycoside extract.

Then, leakage amounts of baicalin in the above prepared calcium carbonate toothpaste with the microcapsule containing the plant glycoside extract and silica toothpaste with the microcapsule were respectively tested. A specific test method included: the toothpaste was stored in a sealed glass bottle, and the glass bottle was placed in a 45° C. constant-temperature oven for an accelerated storage test. 1 g (accurately-weighed) of the toothpaste stored on Day 0 and Day 90 was placed in a centrifuge tube respectively, 10 g of water was added and allowed to stand for half an hour until the toothpaste was dispersed, and the microcapsules were separated from the toothpaste slurry by using a mesh screen with a pore diameter of 100 μm and collected in the centrifuge tube. The microcapsules were put in an ethanol-phosphoric acid aqueous solution (a volume ratio of anhydrous ethanol, phosphoric acid, and water being=50:6:44), and heated for 15 min at 85° C., to cause wall materials of the microcapsules to be completely melted and core materials to be completely dissolved. Vortex oscillation was performed for 2 min after the temperature was rapidly cooled to room temperature, ultrasound extraction was performed for 40 min in a 25° C. water bath, centrifugation was performed for 10 min at 5000 rpm, and supernatant was made to 25 mL by using the ethanol-phosphoric acid aqueous solution. A 0.22 μm filter membrane was used for filtration, and filtrate was taken as a sample solution. A baicalin content was determined through high performance liquid chromatography, and a leakage rate of the baicalin was calculated.

$$Leakage rate ( \% ) = ( 1 - \frac{\begin{matrix}Content of baicalin remained \\ in microcapsule in 1 g of toothpaste\end{matrix}}{\begin{matrix}Total content of baicalin in \\ microcapsule added to 1 g of toothpaste\end{matrix}} ) \times 100 \% .$$

Leakage rate data of the baicalin in the calcium carbonate toothpaste and silica toothpaste measured according to the above test methods was recorded in Table 1 below.

TABLE 1
Leakage rate of baicalin in calcium carbonate
toothpaste and silica toothpaste (%, n = 3)

| | | | Calcium carbonate | |
|---|---|---|---|---|
| | Silica toothpaste system | | toothpaste system | |
| | Day 0 | Day 90 | Day 0 | Day 90 |
| Embodiment 1 | 54.31 ± 0.57 | 57.73 ± 1.75 | 77.10 ± 1.45 | 96.25 ± 0.03 |
| Embodiment 2 | 50.83 ± 0.26 | 52.53 ± 1.41 | 70.08 ± 1.78 | 87.59 ± 0.39 |
| Embodiment 3 | 32.86 ± 0.07 | 34.60 ± 1.87 | 52.59 ± 3.40 | 65.75 ± 1.54 |
| Embodiment 4 | 28.68 ± 2.35 | 29.54 ± 3.27 | 40.62 ± 1.99 | 64.67 ± 2.06 |
| Embodiment 5 | 22.36 ± 1.03 | 28.65 ± 0.25 | 28.21 ± 0.46 | 50.89 ± 0.78 |
| Embodiment 6 | 24.78 ± 0.47 | 27.57 ± 0.13 | 33.82 ± 3.01 | 71.55 ± 0.91 |
| Embodiment 7 | 34.37 ± 2.46 | 36.79 ± 1.55 | 40.74 ± 7.89 | 74.68 ± 2.31 |
| Embodiment 8 | 24.65 ± 1.72 | 31.45 ± 3.11 | 50.98 ± 1.59 | 99.01 ± 0.27 |
| Embodiment 9 | 34.53 ± 0.72 | 35.95 ± 0.44 | 60.20 ± 7.28 | 99.51 ± 0.25 |
| Embodiment 10 | 42.65 ± 0.69 | 48.36 ± 0.47 | 56.49 ± 4.35 | 77.70 ± 0.01 |
| Embodiment 11 | 31.70 ± 0.92 | 39.24 ± 1.13 | 40.12 ± 4.78 | 57.61 ± 0.59 |
| Embodiment 12 | 23.63 ± 3.68 | 23.65 ± 1.81 | 17.97 ± 1.79 | 44.23 ± 2.01 |
| Embodiment 13 | 15.34 ± 2.41 | 20.02 ± 0.22 | 15.13 ± 1.31 | 39.50 ± 2.32 |
| Comparative | 70.83 ± 0.16 | 92.53 ± 1.31 | 75.08 ± 1.18 | 95.59 ± 0.59 |
| example 1 | | | | |

From Table 1, it might be learned that compared with Comparative example 1, the microcapsules containing the plant glycoside extracts in Embodiments 1-13 of the present disclosure are more stable in the silica toothpaste that those in the calcium carbonate toothpaste. By comparing Embodiments 1-13, it might be learned that the leakage rates of the baicalin all reduced with the increasing of sizes of the pill cores, the increasing of the carnauba proportion in the coating material led to an increase in the leakage rates of the baicalin, and the gaining of coating weight led to a reduction in the leakage rate of the microcapsules in the toothpaste.

Then, the release rates of the calcium carbonate toothpaste and silica toothpaste of the above prepared microcapsules containing the plant glycoside extracts during simulated tooth brushing were respectively tested. A specific test method included: 1 g of the toothpaste was taken, tooth brushing was simulated for 3 min on resin dentures, and a consistent force was maintained during the brushing of the toothpaste. The resin dentures were washed with water after brushing, and all toothpaste slurry was collected. The microcapsules still remained in the toothpaste slurry were separated by using the mesh screen with a pore diameter of 100 μm. The remaining microcapsules were put in the ethanol-phosphoric acid aqueous solution (the volume ratio of anhydrous ethanol, phosphoric acid, and water being=50:6:44), and heated for 15 min at 85° C., to cause the wall materials to be completely melted and the core materials to be completely dissolved. Vortex oscillation was performed for 2 min after the temperature was rapidly cooled to room temperature, ultrasound extraction was performed for 40 min in a 25° C. water bath, centrifugation was performed for 10 min at 5000 rpm, and supernatant was made to 25 mL by using the ethanol-phosphoric acid aqueous solution. A 0.22 μm filter membrane was used for filtration, and filtrate was taken as a sample solution. The baicalin content was determined through high performance liquid chromatography, a simulated release rate and a tooth brushing release increment were calculated.

$$Simulated release rate ( \% ) = ( 1 - \frac{\begin{matrix}Content of baicalin remained \\ in microcapsule in toothpaste slurry\end{matrix}}{\begin{matrix}Total content of baicalin of \\ microcapsule added to toothpaste\end{matrix}} ) \times 100 \% .$$

$$Tooth brushing release increment ( \% ) = simulated release rate ( \% ) - leakage amount ( \% )$$

Release rate data of the baicalin in the calcium carbonate toothpaste and silica toothpaste measured according to the above test methods was recorded in Table 2 below.

TABLE 2
Simulated release rate and tooth brushing release increment of baicalin
in calcium carbonate toothpaste and silica toothpaste (%, n = 3)

| | | | Calcium carbonate | |
|---|---|---|---|---|
| | Silica toothpaste system | | toothpaste system | |
| | Simulated | Tooth brushing | Simulated | Tooth brushing |
| | release rate | release increment | release rate | release increment |
| Embodiment 1 | 75.90 ± 1.76 | 21.60 ± 0.91 | 98.32 ± 0.74 | 21.22 ± 1.30 |
| Embodiment 2 | 68.51 ± 0.65 | 17.69 ± 1.10 | 93.07 ± 2.58 | 22.99 ± 0.67 |
| Embodiment 3 | 58.15 ± 1.02 | 23.29 ± 4.56 | 87.29 ± 1.30 | 34.70 ± 2.09 |
| Embodiment 4 | 54.98 ± 1.75 | 26.29 ± 1.27 | 71.75 ± 1.05 | 31.12 ± 0.95 |
| Embodiment 5 | 53.12 ± 1.51 | 30.76 ± 1.77 | 65.19 ± 3.02 | 36.98 ± 3.25 |
| Embodiment 6 | 57.68 ± 10.68 | 32.91 ± 3.53 | 68.71 ± 1.69 | 34.89 ± 4.33 |
| Embodiment 7 | 58.96 ± 1.52 | 24.50 ± 1.15 | 70.69 ± 3.75 | 29.96 ± 1.35 |
| Embodiment 8 | 47.81 ± 3.18 | 23.16 ± 3.38 | 67.53 ± 3.01 | 16.54 ± 1.44 |
| Embodiment 9 | 37.34 ± 5.50 | 1.81 ± 0.47 | 63.85 ± 0.64 | 3.64 ± 0.99 |
| Embodiment 10 | 75.43 ± 2.05 | 32.78 ± 2.75 | 79.14 ± 1.12 | 22.6 ± 51.8 |
| Embodiment 11 | 59.87 ± 3.67 | 28.17 ± 1.08 | 73.19 ± 1.43 | 33.07 ± 3.22 |
| Embodiment 12 | 52.45 ± 8.32 | 28.82 ± 3.86 | 58.69 ± 1.75 | 40.72 ± 2.04 |
| Embodiment 13 | 44.42 ± 4.55 | 29.07 ± 1.47 | 49.78 ± 5.06 | 34.66 ± 2.29 |
| Comparative | 88.51 ± 0.65 | 17.68 ± 0.70 | 91.07 ± 2.58 | 15.99 ± 0.57 |
| example 1 | | | | |

As shown in Table 2, by comparing Embodiments 1-6, it might be learned that the increasing of the particle size of the microcapsule containing the plant glycoside extract might increase the tooth brushing release increment of the microcapsule; by comparing Embodiments 7-9, it might be learned that the increasing of the proportion of the carnauba in the coating material might reduce the tooth brushing release increment of the microcapsule containing the plant glycoside extract; and by comparing Embodiments 10-13, it might be learned that the reduction of coating weight gained might increase the tooth brushing release increment of the microcapsule containing the plant glycoside extract.

### (3) Test for Storage Stability of Baicalin

1 g of the microcapsule containing the plant glycoside extract prepared in Embodiment 12 and 39 g of the silica toothpaste substrate or 39 g of the calcium carbonate toothpaste substrate were stirred for 1 min at a rotary speed of 50 rpm for well mixing, so as to obtain the silica toothpaste with the microcapsule containing the plant glycoside extract and the calcium carbonate toothpaste with the microcapsule containing the plant glycoside extract as an implementation group, and the toothpastes in the implementation group both were microencapsulated plant glycoside extracts. An equivalent amount of the plant glycoside extract was additionally taken and uniformly mixed and stirred with 39 g of the silica toothpaste substrate or 39 g of the calcium carbonate toothpaste substrate as a control group, and the toothpastes in the control group were non-microencapsulated plant glycoside extracts. The prepared toothpastes were respectively placed in the 45° C. constant-temperature oven for storage for 3 months in a sealed manner. Proper amounts of samples for the silica toothpaste with the microcapsule containing the plant glycoside extract and the calcium carbonate toothpaste with the microcapsule containing the plant glycoside extract were taken on Day 0, 1, 5, 10, 15, 30, 60, and 90 during accelerated storage and placed in a mortar, and the microcapsules were ground to completely break. 1 g (accurate to 0.1 mg) of the ground toothpaste sample were weighed and put in a 50 mL stoppered centrifuge tube, 1 g of quartz sand and 20 mL of the ethanol monophosphate solution (in the ethanol-phosphoric acid solution (the volume ratio of the anhydrous ethanol, the phosphoric acid, and the water being 50:6:44), the mass was recorded, then vortex oscillation was performed for 2 min, ultrasound extraction was performed for 40 min in the 25° C. water bath and then the temperature was cooled to room temperature, and then a mass loss was compensated by using the above ethanol-phosphoric acid solution. Centrifugation was performed for 10 min at 5000 rpm, and the supernatant was filtered by using a 0.22 μm filter membrane. The baicalin content was determined through high performance liquid chromatography, and retention rates of the baicalin at different accelerated storage periods were calculated according to a total content of the baicalin in 1 g of the toothpaste.

$$Retention rate of baicalin on Day N ( \% ) = ( 1 - \frac{Baicalin content in toothpaste on Day N}{Baicalin content in toothpaste on Day 0} ) \times 100 \%$$

Retention rate data of the baicalin in the toothpaste in the implementation group and control group determined according to the above test method was shown in Table 3 below.

TABLE 3
Retention rate of baicalin in toothpaste during accelerated
storage at 45° C. (%, n = 3)

| 45° C. | | | Calcium carbonate | |
|---|---|---|---|---|
| storage | Silica toothpaste system | | toothpaste system | |
| duration | Implementation | | Implementation | |
| (day) | group | Control group | group | Control group |
| 0 | 99.71 ± 0.07 | 98.65 ± 1.35 | 99.61 ± 0.21 | 98.27 ± 0.52 |
| 5 | 98.51 ± 0.02 | 93.75 ± 0.02 | 96.18 ± 1.37 | 83.80 ± 1.10 |
| 10 | 95.00 ± 0.41 | 80.31 ± 1.45 | 91.45 ± 0.76 | 69.12 ± 0.25 |
| 15 | 95.09 ± 0.33 | 79.74 ± 0.40 | 89.35 ± 0.46 | 66.35 ± 0.17 |
| 30 | 93.54 ± 1.06 | 74.97 ± 0.76 | 84.45 ± 1.50 | 52.13 ± 0.21 |
| 60 | 90.89 ± 0.96 | 69.57 ± 0.53 | 77.21 ± 1.62 | 38.47 ± 0.18 |
| 90 | 89.34 ± 0.12 | 65.23 ± 2.22 | 70.84 ± 1.02 | 27.53 ± 0.40 |

As shown in Table 3, in the calcium carbonate toothpaste and the silica toothpaste, microencapsulation embedding caused the retention rates of the baicalin after 90 days of accelerated storage to be increased by 43.32% and 24.11% compared with those in the control group, indicating that microencapsulation facilitated the retention of the baicalin during toothpaste storage.

1 g of each of the microcapsules containing the plant glycoside extracts prepared in Comparative examples 2-3 were taken and stirred with 39 g of the silica toothpaste substrate for 1 min at a rotary speed of 50 rpm for well mixing, so as to obtain the silica toothpaste with the microcapsule containing the plant glycoside extract, and the prepared toothpastes were respectively placed in the 45° C. constant-temperature oven for storage for 1 months in a sealed manner. A proper amount of a sample for the silica toothpaste with the microcapsule containing the plant glycoside extract was taken on Day 0, 2, 4, 7, 10, 20, and 30 during accelerated storage and placed in a mortar, and the microcapsules were ground to completely break. 1 g (accurate to 0.1 mg) of the ground toothpaste sample were weighed and put in a 50 mL stoppered centrifuge tube, 1 g of quartz sand and 20 mL of the ethanol monophosphate solution (in the ethanol-phosphoric acid solution (the volume ratio of the anhydrous ethanol, the phosphoric acid, and the water being 50:6:44), the mass was recorded, then vortex oscillation was performed for 2 min, ultrasound extraction was performed for 40 min in the 25° C. water bath and then the temperature was cooled to room temperature, and then a mass loss was compensated by using the above ethanol-phosphoric acid solution. Centrifugation was performed for 10 min at 5000 rpm, and the supernatant was filtered by using a 0.22 μm filter membrane. The baicalin content was determined through high performance liquid chromatography, and leakage rates of the baicalin at different accelerated storage periods were calculated according to a total content of the baicalin in 1 g of the toothpaste. Leakage rate data of the baicalin in the toothpaste in Comparative examples 2-3 determined according to the above test method was shown in Table 4 below.

TABLE 4
Leakage rates of baicalin in toothpaste
in Comparative examples 2-3

| | | |
|---|---|---|
| 45° C. | Comparative | Comparative |
| leakage rate | example 2 | example 3 |

| | | | |
|---|---|---|---|
| 0 | day | 15.09 ± 0.15 | 25.27 ± 0.40 |

| | | |
|---|---|---|
| Day 2 | 75.39 ± 0.20 | 73.02 ± 0.28 |

| | | | |
|---|---|---|---|
| 4 | days | 77.78 ± 0.78 | 76.43 ± 0.67 |
| 7 | days | 79.98 ± 0.77 | 83.05 ± 0.88 |
| 10 | days | 80.69 ± 0.76 | 89.74 ± 1.83 |
| 20 | days | 90.33 ± 0.44 | 90.27 ± 0.20 |
| 30 | days | 91.15 ± 0.65 | 93.51 ± 1.71 |

From Table 4, it might be learned that the microcapsules in Comparative examples 2-3 were stored in the silica toothpaste for 30 days in an accelerated manner, the leakage rate of the baicalin thereof had exceeded 90%, and storage stability was significantly lower than that of the microcapsule in the present disclosure. The reasons were that, water content in the silica toothpaste was relatively high, the content of hydrophilic components such as a humectant was much higher than that in the calcium carbonate toothpaste, effects of hydrophilic resistant ingredients in the microcapsules in Comparative examples 2-3 were poor, and the leakage rate in the silica toothpaste was high. The retention rate of the microcapsule in the present disclosure after 90 days of accelerated storage still exceeded 70%, that is, the leakage rate was not higher than 30%, such that, in regardless of silica toothpaste or calcium carbonate toothpaste, the microcapsule in the present disclosure had better resistance to the action of hydrophilic ingredients, and had excellent storage stability.

### (4) Test for Mouth Ulcer Treatment Effect

A mouth ulcer model was constructed for SPF-grade SD rats weighing 230-250 g at a weekly age of about 6 weeks. After anesthesia of animals in the modeling group, a filter paper with a diameter of 0.4 mm was soaked in a 50% NaOH solution, and then pressed on oral maxillary mucosa for ulcer modeling. The animals for mouth ulcer modeling were grouped into a model control group, a positive control group, and an experimental group. The model control group used normal saline to coat an ulcer place; the positive control group used mirabilitum praeparatum as a positive control; and the experimental group was grouped into a microencapsulation group and a non-microencapsulation group. In the microencapsulation group, 1 g of the microcapsule containing the plant glycoside extract obtained in Embodiment 12 and 39 g of the calcium carbonate toothpaste substrate were mixed; in the non-microencapsulation group, 0.2 g of plant glycoside extract powder and 30.15 g of the calcium carbonate toothpaste substrate were mixed; and baicalin contents in the toothpaste in the microencapsulation group and the non-microencapsulation group were the same. After the experiment toothpastes in the two groups were subjected to an accelerated storage experiment at 45° C. for 0, 60 and 90 days, a rat mouth ulcer healing experiment was performed.

Administration started after 24 h of modeling, after isoflurane anesthesia of the animals in each administration group, 0.2 g of the corresponding toothpaste was smeared at the ulcer place in each experimental group, and the toothpaste in the microencapsulation group was completely ground and broken by using the mortar before administration; 0.03 g of the mirabilitum praeparatum was smeared in the positive control group; the normal saline was smeared in the model control group; and after maintaining for 3 min, test substances were removed. An administration frequency was 2 times/day, once in the morning and once in the afternoon at a fixed time, for 8 consecutive days. A diameter of an ulcer surface is measured during the period, an ulcer area was calculated, and a mouth ulcer healing rate was calculated according to changes in the ulcer area. Mouth ulcer healing rate data of the rates measured according to the above test method was shown in Table 5 below.

TABLE 5
Mouth ulcer healing rate of rats

| | Day 5 of | Day 8 of |
|---|---|---|
| | administration | administration |
| Negative control group | 33.38 ± 6.45 | 64.75 ± 6.13 |
| Positive control group | 46.42 ± 7.35 | 78.99 ± 3.37 |
| Non-microencapsulation-Day 0 of | 46.97 ± 3.85 | 83.62 ± 3.99 |
| accelerated storage | | |
| Non-microencapsulation-Day 60 of | 28.07 ± 1.83 | 77.00 ± 2.91 |
| accelerated storage | | |
| Non-microencapsulation-Day 90 of | 28.95 ± 6.26 | 69.95 ± 3.56 |
| accelerated storage | | |
| Microencapsulation-Day 0 of | 45.74 ± 7.43 | 81.40 ± 5.83 |
| accelerated storage | | |
| Microencapsulation-Day 60 of | 35.17 ± 3.00 | 84.20 ± 3.35 |
| accelerated storage | | |
| Microencapsulation-Day 90 of | 50.98 ± 5.04 | 80.96 ± 3.94 |
| accelerated storage | | |

As shown in Table 5, compared to direct addition of the plant glycoside extract to the toothpaste, microencapsulation embedding could ensure that, after being subjected to accelerated storage in the toothpaste, glycoside active ingredients still had good effect of promoting the healing of mouth ulcer.

To sum up, the present disclosure used the natural wax as the coating material, and the plant glycoside extract was encapsulated into the microcapsule. The microcapsule was smooth in surface and compact in encapsulation. Therefore, the stability of glycoside substances is improved, unstable phenomena such as oxidative decomposition were avoided, and toothpastes containing the microcapsule had longer storage stability while the stability of the plant glycoside extract in the toothpastes was improved. Furthermore, in the present disclosure, by adjusting the component of the natural wax and component proportions, the mechanical property of the shell of the microcapsule could be changed to reduced breakage and leakage due to friction and wear of the microcapsule during paste preparation, such that most microcapsules were not broken during paste preparation, and it could also ensure that the active ingredients of the plant glycoside extract could be released during tooth brushing when the microcapsule was used in the toothpaste, thereby achieving effects of treating mouth ulcer and the like.

The above embodiments of the present disclosure are described in detail, but the present disclosure is not limited to the above embodiments, and within the scope of the knowledge possessed by those of ordinary skill in the art, a variety of changes may also be made without departing from the premise of the purpose of the present disclosure. In addition, in the case of no conflict, the embodiments of the present disclosure and features in the embodiments may be combined with one another without conflict.

What is claimed is:

1. A plant glycoside microcapsule, wherein the microcapsule has a core-shell structure, and a shell of the microcapsule comprises a natural wax; a core of the microcapsule comprises a plant glycoside extract; a mass ratio of the core of the microcapsule to the shell of the microcapsule is 1:(0.3-0.7); and the natural wax is a mixture of beeswax and carnauba in a mass ratio of 1:(0.05-0.2).

2. The microcapsule according to claim 1, wherein raw materials of the plant glycoside extract are selected from at least one of scutellaria baicalensis, honeysuckle flower, coptis chinensis, licorice, rhizoma bolbostemmae, herba eupatorii, pogostemon cablin, tangerine peel, rhizoma zingiberis, chinese date, rhizoma anemarrhenae, cortex phellodendri, cortex moutan, radix rehmanniae, or radix ophiopogonis.

3. The microcapsule according to claim 1, wherein the core of the microcapsule further comprises a filling agent and a disintegrating agent; and a mass ratio of the plant glycoside extract, the filling agent, and the disintegrating agent is 1:(1-1.25):(0.05-0.3).

4. The microcapsule according to claim 3, wherein the disintegrating agent is selected from at least one of croscarmellose or carboxymethyl starch; and/or
the filling agent is selected from microcrystalline cellulose.

5. The microcapsule according to claim 1, wherein a particle size of the microcapsule is 370-920 μm; and/or
a content of baicalin in the microcapsule is 5-6.3%; and/or
a coating weight gained in the microcapsule is 27.07%-57.79%.

6. The microcapsule according to claim 2, wherein a particle size of the microcapsule is 370-920 μm; and/or
a content of baicalin in the microcapsule is 5-6.3%; and/or
a coating weight gained in the microcapsule is 27.07%-57.79%.

7. The microcapsule according to claim 3, wherein a particle size of the microcapsule is 370-920 μm; and/or
a content of baicalin in the microcapsule is 5-6.3%; and/or
a coating weight gained in the microcapsule is 27.07%-57.79%.

8. The microcapsule according to claim 4, wherein a particle size of the microcapsule is 370-920 μm; and/or
a content of baicalin in the microcapsule is 5-6.3%; and/or
a coating weight gained in the microcapsule is 27.07%-57.79%.

9. A method for preparing the microcapsule according to claim 1, wherein the preparation method comprises the following steps:
S1: preparing a core material of a microcapsule into a pill core; and
S2: coating the pill core by using a natural wax, so as to prepare the microcapsule.

10. The method for preparing the microcapsule according to claim 9, wherein a particle size of the pill core is 0.3-0.7 mm.

11. A toothpaste, comprising a plant glycoside microcapsule, the microcapsule has a core-shell structure, and a shell of the microcapsule comprises a natural wax; a core of the microcapsule comprises a plant glycoside extract; a mass ratio of the core of the microcapsule to the shell of the microcapsule is 1:(0.3-0.7); and the natural wax is a mixture of beeswax and carnauba in a mass ratio of 1:(0.05-0.2).

12. The toothpaste according to claim 11, wherein raw materials of the plant glycoside extract are selected from at least one of scutellaria baicalensis, honeysuckle flower, coptis chinensis, licorice, rhizoma bolbostemmae, herba eupatorii, pogostemon cablin, tangerine peel, rhizoma zingiberis, chinese date, rhizoma anemarrhenae, cortex phellodendri, cortex moutan, radix rehmanniae, or radix ophiopogonis.

13. The toothpaste according to claim 11, wherein the core of the microcapsule further comprises a filling agent and a disintegrating agent; and a mass ratio of the plant glycoside extract, the filling agent, and the disintegrating agent is 1:(1-1.25):(0.05-0.3).

14. The toothpaste according to claim 13, wherein the disintegrating agent is selected from at least one of croscarmellose or carboxymethyl starch; and/or
the filling agent is selected from microcrystalline cellulose.

15. The toothpaste according to claim 11, wherein a particle size of the microcapsule is 370-920 μm; and/or
a content of baicalin in the microcapsule is 5-6.3%; and/or
a coating weight gained in the microcapsule is 27.07%-57.79%.

16. The toothpaste according to claim 12, wherein a particle size of the microcapsule is 370-920 μm; and/or
a content of baicalin in the microcapsule is 5-6.3%; and/or
a coating weight gained in the microcapsule is 27.07%-57.79%.

17. The toothpaste according to claim 13, wherein a particle size of the microcapsule is 370-920 μm; and/or
a content of baicalin in the microcapsule is 5-6.3%; and/or
a coating weight gained in the microcapsule is 27.07%-57.79%.

18. The toothpaste according to claim 14, wherein a particle size of the microcapsule is 370-920 μm; and/or
a content of baicalin in the microcapsule is 5-6.3%; and/or
a coating weight gained in the microcapsule is 27.07%-57.79%.

19. Use of the microcapsule according to claim 1 in preparation of oral care products.
